(19) United States
(12) Patent Application Publication
(10) Pub. No.: US 2024/0417381 A1
(43) Pub. Date: Dec. 19, 2024

(54) AN IMPROVED PROCESS FOR THE PREPARATION OF NITAZOXANIDE AND INTERMEDIATES THEREOF

(71) Applicant: COUNCIL OF SCIENTIFIC AND INDUSTRIAL RESEARCH AN INDIAN REGISTERED BODY INCORPORATED, New Delhi, Delhi (IN)

(72) Inventors: Sasidhar Balappa SOMAPPA, Kerala Thiruvananthapuram (IN); Praveen Kumar VALMIKI, Kerala Thiruvananthapuram (IN); Basavaraja DURUGAPPA, Kerala Thiruvananthapuram (IN)

(73) Assignee: COUNCIL OF SCIENTIFIC AND INDUSTRIAL RESEARCH AN INDIAN REGISTERED BODY INCORPORATED, New Delhi, Delhi (IN)

(21) Appl. No.: 18/697,988

(22) PCT Filed: Sep. 21, 2022
(86) PCT No.: PCT/IN2022/050843

§ 371 Date: Apr. 2, 2024

(30) Foreign Application Priority Data
Oct. 5, 2021 (IN) 202111045217

(51) Int. Cl. C07D 277/58

(52) U.S. Cl. CPC C07D 277/58

(57) ABSTRACT

The present invention relates to the process for the preparation of Nitazoxanide and intermediates thereof. The invention explores readily available starting materials with reaction conditions that are suitable for industrial scale applications. The present invention discloses, a single vessel process to prepare 5-nitrothiazol-2-amine starting from a very basic starting material 1,1-dimethoxy-N, N-dimethylmethanamine. Overall intermediates throughout the process is free from column chromatography and the final product Nitazoxanide is purified by precipitation. All the synthesized compounds were confirmed by various spectroscopic techniques such as Fourier transform infrared spectroscopy, ¹H NMR, ¹³C NMR, HRMS and elemental analysis.

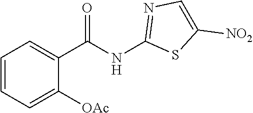 [SMILES: CC(=O)Oc1ccccc1C(=O)Nc1ncc([N+](=O)[O-])s1]

### FIELD OF THE INVENTION

The present invention relates to the process for the preparation of Nitazoxanide and intermediates thereof. More particularly the invention explores readily available starting materials and reaction conditions that are suitable for industrial-scale applications. The synthesized compounds were confirmed by various spectroscopic techniques such as fourier transform infrared spectroscopy, ¹H NMR, ¹³C NMR, ¹⁹F NMR spectroscopy, HRMS and single-crystal X-ray analysis.

### BACKGROUND OF THE INVENTION

Nitazoxanide is a 5-nitrothiazol analogue with anti-parasitic and antibacterial activity with a broad spectrum action. Its chemical formula is 2-acetolyloxy-N-(5-nitro-2-tiazolyl)benzamide and has the following structure (Formula I):

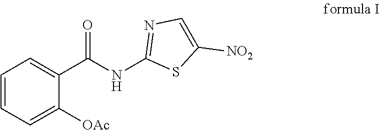 [SMILES: CC(=O)Oc1ccccc1C(=O)Nc1ncc([N+](=O)[O-])s1]

Nitazoxanide is currently approved in several countries to treat infections caused by parasites in two pharmaceutical forms: immediate-release tablets and powder for immediate release suspension. Nitazoxanide is also found to have antiviral activity against different viral infections such as coronaviruses, influenza, hepatitis C virus (HCV), hepatitis B virus (HBV), and other viruses, signifying its potential as a broad-spectrum antiviral drug. Very recently, it has been reported that Nitazoxanide exhibited in vitro inhibition of SARS-CoV-2 at a small micromolar concentration. It suppresses the production of cytokines emphasizing its potential to manage COVID-19-induced cytokine storm.

Reference may be made to patent application U.S. Pat. No. 3,950,351, wherein the process for the synthesis of Nitazoxanide and the use thereof disclosed first time.

Reference may be made to patent application U.S. Pat. Nos. 5,578,621 and 5,387,598, wherein Nitazoxanide formulations suitable for treating parasitic intestinal infections through surfactant excipients and starch were disclosed

Reference may be made to patent application U.S. Pat. No. 5,968,961, wherein Nitazoxanide formulations with optimal particle size led to the improved therapeutic effect and reduced the side effects disclosed.

Reference may be made to patent application U.S. Pat. No. 6,117,894, wherein improved nitazoxanide formulations in terms of stability through the use of acidifying excipients disclosed.

Reference may be made to patent application US 20100209505, wherein Nitazoxanide compositions in double-coated tablets disclosed in which one coating is of immediate release and the other coating is a controlled-release coating specifically designed for the treatment of chronic viral hepatitis C.

Reference may be made to patent application U.S. Pat. No. 3,950,351, wherein the reaction of benzoyl chloride and 2-amino-5-nitro-thiazole in anhydrous tetrahydrofuran and triethylamine disclosed. The reaction mixture, which became slightly warm, was stirred for 45 minutes and then poured under agitation into distilled water. The stirring was continued until the precipitation of the desired compound was complete. The obtained precipitate was dried, washed with water, dried again, and recrystallized from methanol. The process for the synthesis of Nitazoxanide of formula I as well as derivatives is represented schematically as follows.

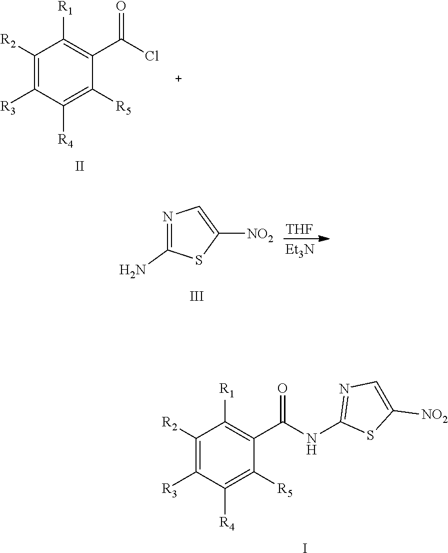

in which R₁═OAc; R₂═R₃=R₄═R₅═H

Reference may be made to patent application CN101007792A, wherein direct reaction of acetylsalicylic acid and 2-amino-5-nitro-thiazole in acetone in the presence of DCC and triethylamine (Et₃N) at −2 to −3° C. for 3 to 6 hrs disclosed.

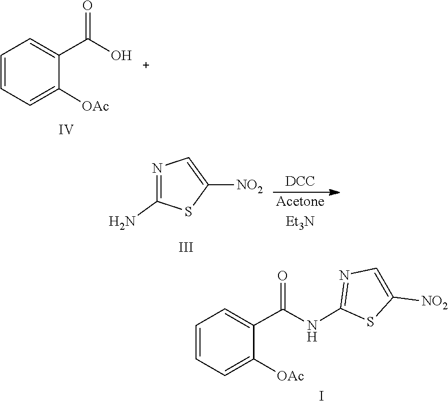

Reference may be made to patent application CN101602744B, wherein the reaction of acetylsalicylic chloride in acetone and 2-amino-5-nitro-thiazole in acetone are reacted together at 24 to 26° C. in the presence of Et₃N disclosed. The crude product was subjected for some lengthy procedure to purify the final product.

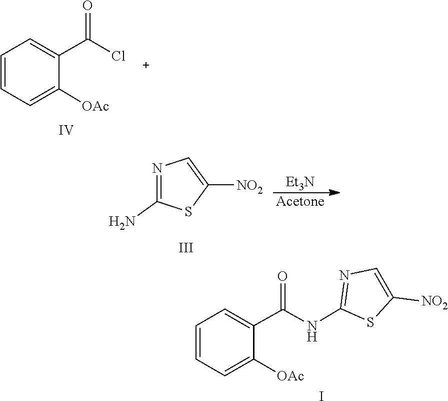

Reference may be made to patent application CN103159697A, wherein process comprising the reaction of acetylsalicylic acid with thionyl chloride at 50° C.-70° C. so as to generate O-Acetylsalicyloyl chloride, then resulting O-Acetylsalicyloyl chloride was dissolved in 1, 4-dioxane or N, N-dimethyl formamide (DMF), and react with 2-amino-5-nitrothiazole in the presence of 1, 4-dioxane or N, N-dimethyl formamide and triethylamine at room temperature disclosed. After the completion of the reaction, the product Nitazoxanide is isolated by following the procedure reported. The schematic representation is as shown in below.

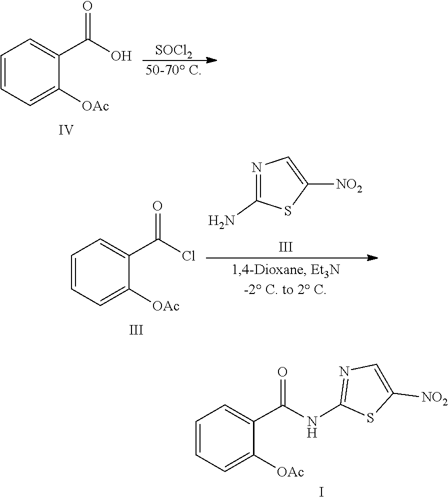

Reference may be made to patent application CN104447613A, wherein direct reaction of acetylsalicylic acid and 2-amino-5-nitro-thiazole in DCE at an elevated temperature of 100° C. disclosed.

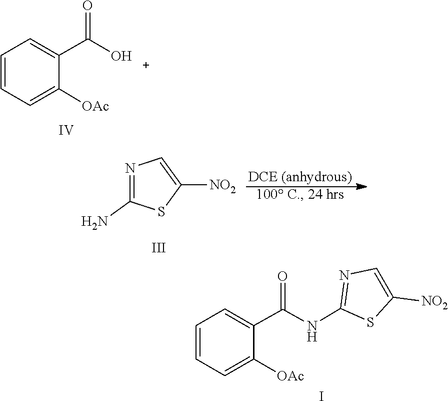

In general, the existing prior art utilizes the advanced commercially available starting materials, acetylsalicylic chloride (II) or acetylsalicylic acid (IV) and 2-amino-5-nitro-thiazole (III). The purity and the yield of Nitazoxanide as per the disclosed processes are not satisfactory, and also the said process involves multistep synthesis for the preparation of 2-amino-5-nitro-thiazole (III). The stated techniques are tedious, labor-intensive, and time-consuming, not suitable for industrial-scale, increasing manufacturing costs. Moreover, the said process involves the use of reagents that form certain impurities and results in the formation of the final compound with a lesser purity.

In view of intrinsic fragility, there is a need in the art to develop a simple, industrially feasible, and scalable process for the synthesis of Nitazoxanide that would avoid the aforementioned difficulties. Moreover, it becomes necessary to prepare highly pure intermediates to prepare pure Nitazoxanide.

The present invention discloses the improved process for the preparation of Nitazoxanide by involving either of single vessel process, selection of reaction solvents or precipitation/crystallization techniques, which are useful for the scale up process while retaining the chemical purity of the product.

### Objectives of the Invention

An objective of the present invention is to develop an improved and industrially feasible process for Nitazoxanide. The invention focused majorly on the process modifications, which are feasible for scale up process.

An objective of the present invention is to develop an industrially viable and cost-effective process for the preparation of Nitazoxanide.

Yet another objective of the present invention is developing a single-vessel process to prepare a key starting material, 5-nitrothiazol-2-amine.

Yet another objective of the present invention is to develop a column chromatography-free process for the preparation of Nitazoxanide.

### SUMMARY OF THE PRESENT INVENTION

The present invention encompasses an improved process for the preparation of Nitazoxanide of formula I, also known as 2-((5-nitrothiazol-2-yl)carbamoyl)phenyl acetate;

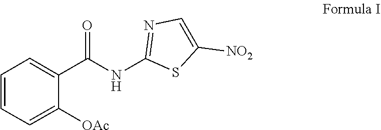 [SMILES: CC(=O)Oc1ccccc1C(=O)Nc1ncc([N+](=O)[O-])s1]

comprising the steps of:
a) reacting 1,1-dimethoxy-N,N-dimethylethanolamine

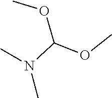

with nitromethane in the presence of an organic solvent or neat to obtain N, N-dimethyl-2-nitroethenamine,
b) reacting N, N-dimethyl-2-nitroethenamine

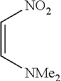

with the brominating reagent in an organic solvent to obtain 2-bromo-N, N-dimethyl-2-nitroethenamine,
c) the cyclo condensation of resulting compound 2-bromo-N, N-dimethyl-2-nitroethenamine

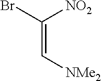

with thiourea in an organic solvent and an acid catalyst to obtain 5-nitrothiazol-2-amine of Formula II,

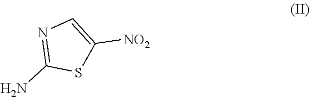

d) condensing the resultant compound of Formula II with 2-acetoxybenzoic acid of Formula III

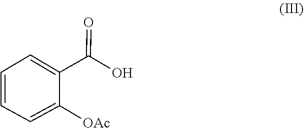 [SMILES: CC(=O)Oc1ccccc1C(=O)O]

in the presence of N—N′-dicyclohexylcarbodiimide (DCC), acetone and triethylamine (Et₃N) as a base to obtain a substantially pure Nitazoxanide of Formula I, and
e) optionally reacting the 2-acetoxybenzoic acid of Formula III,

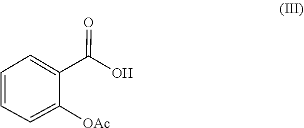 [SMILES: CC(=O)Oc1ccccc1C(=O)O]

with a reagent in an organic solvent to obtain 2-(chlorocarbonyl)phenyl acetate of Formula IV,

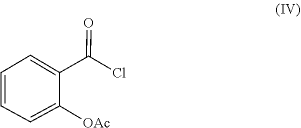 [SMILES: CC(=O)Oc1ccccc1C(=O)Cl]

this upon condensing with 5-nitrothiazol-2-amine of Formula II in the presence of tetrahydrofuran (THF) and triethylamine (Et₃N) as a base to obtain a substantially pure Nitazoxanide of Formula I.

The present disclosure further relates to invention, wherein in step a) the solvent is selected from the group consisting of tetrahydrofuran (THF), dichloromethane (DCM), ethylene dichloride (DCE), chloroform, benzene, and mixtures thereof, or neat.

The present disclosure further relates to invention, wherein the brominating reagent in step b) is selected from the group consisting of bromine or N-bromosuccinimide.

The present disclosure further relates to invention, wherein the solvent in step b) is selected from the group consisting of tetrahydrofuran (THF), dichloromethane (DCM), ethylene dichloride (DCE), chloroform, benzene and mixtures thereof and the catalyst in step c) is acetic acid, sulphuric acid or hydrochloric acid.

The present disclosure further relates to invention, wherein solvent in step c) is selected from the group consisting of dimethylformamide (DMF), dimethyl sulfoxide (DMSO), dimethylacetamide (DMAc), and mixtures thereof and temperature in step c) to convert 2-bromo-N, N-dimethyl-2-nitroethenamine into 5-nitrothiazol-2-amine of Formula II is at room temperature.

The present disclosure further relates to invention, wherein the solvent in step e) converts the compound of Formula III to Formula IV, is selected from the group consisting of tetrahydrofuran (THF), dichloromethane (DCM), acetone, Dichloroethane (DCE).

Another aspect of the present invention is to develop a single-vessel process for the preparation of a key starting material, 5-nitrothiazol-2-amine.

Yet another aspect of the present invention is to develop a column chromatography free process for the preparation of Nitazoxanide.

The various features of novelty that characterize the invention are pointed out with particularity in the claims annexed and forming a part of this disclosure. For a better understanding of the present invention, its operating advantages, and specific objectives attained by its uses, reference is made to the accompanying descriptive material in which preferred embodiments of the invention are illustrated.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### Details of Abbreviations

NMR—Nuclear Magnetic Resonance
HRMS—High resolution mass spectrometry
THF—Tetrahydrofuran
Et₃N—triethylamine
DCC—N, N′-Dicyclohexylcarbodiimide
DCE—Dichloroethane
DMF—N, N-dimethyl formamide
SOCl₂—Thionylchloride

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 depicts schematic representation of improved process for the preparation of Nitazoxanide of Formula I from acetylsalicylic acid, in accordance with an embodiment of the present disclosure.

FIG. 2 depicts schematic representation of improved process for the preparation of Nitazoxanide of Formula I from Acetylsalicyloyl chloride, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature in the range of 0-5° C. should be interpreted to include not only the explicitly recited limits of 0° C.-5° C. but also to include sub-ranges, such as 1-4.9° C., and so forth, as well as individual amounts, within the specified ranges, such as 3.2° C., 4.5° C., and so on.

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

In line with the above objectives, the present invention provides
a process for preparing Nitazoxanide of Formula I;

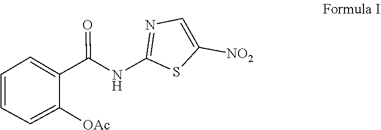 [SMILES: CC(=O)Oc1ccccc1C(=O)Nc1ncc([N+](=O)[O-])s1]

comprising the steps of:
   a) reacting 1,1-dimethoxy-N,N-dimethylethanolamine

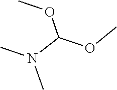

with nitromethane in the presence of an organic solvent or neat to obtain N, N-dimethyl-2-nitroethenamine,
b) reacting N, N-dimethyl-2-nitroethenamine

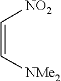

with the brominating reagent in organic solvent to obtain 2-Bromo-N, N-dimethyl-2-nitroethenamine,
c) cyclo condensation of resulting compound 2-Bromo-N, N-dimethyl-2-nitroethenamine

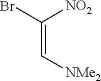

with thiourea in organic solvent and acid catalyst to obtain 5-nitrothiazol-2-amine of Formula II,

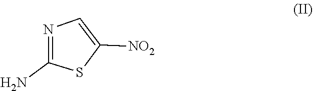

d) condensing the resultant compound of Formula II with 2-acetoxybenzoic acid of Formula III

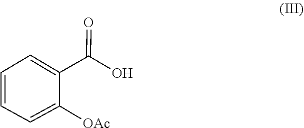 [SMILES: CC(=O)Oc1ccccc1C(=O)O]

in the presence of N—N′-dicyclohexylcarbodiimide (DCC), acetone and triethylamine (Et₃N) as a base to obtain a substantially pure Nitazoxanide of Formula I. and
e) optionally reacting the 2-acetoxybenzoic acid of Formula III,

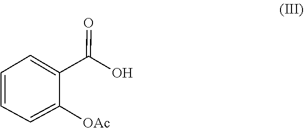 [SMILES: CC(=O)Oc1ccccc1C(=O)O]

with a reagent in the organic solvent to obtain 2-(chlorocarbonyl)phenyl acetate of Formula IV,

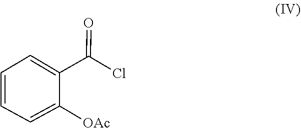 [SMILES: CC(=O)Oc1ccccc1C(=O)Cl]

this upon condensing with 5-nitrothiazol-2-amine of Formula II in the presence of tetrahydrofuran (THF) and triethylamine (Et₃N) as a base to obtain a substantially pure Nitazoxanide of Formula I.

In an embodiment, the present disclosure provides an improved process for the preparation of compound of Formula I as disclosed herein, wherein, in step (a), the organic solvent is selected from the group consisting of tetrahydrofuran (THF), dichloromethane (DCM), ethylene dichloride (DCE), chloroform, benzene, and mixtures thereof, or neat. More particularly, the reaction is carried out in the neat (solvent free) condition.

In an embodiment, the present disclosure provides an improved process for the preparation of compound of Formula I as disclosed herein, wherein the brominating reagent used herein step (b) is selected from the group consisting of bromine or N-bromosuccinimide. More particularly, the brominating reagent is N-bromosuccinimide.

In an embodiment, the present disclosure provides an improved process for the preparation of compound of Formula I as disclosed herein, wherein the organic solvent used in step (b) is selected from the group consisting of tetrahydrofuran (THF), dichloromethane (DCM), ethylene dichloride (DCE), chloroform, benzene, and mixtures thereof. More particularly, the solvent is benzene or chloroform.

In an embodiment, the present disclosure provides an improved process for the preparation of compound of Formula I as disclosed herein, wherein the acid used in step (c) are selected from the group consisting of acetic acid, sulphuric acid or hydrochloric acid; more particularly hydrochloric acid and the solvent used in step (c) is selected from the group consisting of dimethylformamide (DMF), dimethyl sulfoxide (DMSO), dimethylacetamide (DMAc) and mixtures thereof. More particularly the organic solvent is dimethylformamide (DMF). In another embodiment, the step (c) to convert 2-bromo-N, N-dimethyl-2-nitroethenamine into 5-nitrothiazol-2-amine of Formula II is carried out at room temperature.

In an embodiment, the present disclosure provides an improved process for the preparation of compound of Formula I as disclosed herein, wherein in the step (e) of the aforementioned process, the reagent to convert compound of Formula III to compound of Formula IV is oxalyl chloride ((COCl)₂) in dimethylformamide (DMF) and the solvent is selected from the group consisting of tetrahydrofuran (THF), dichloromethane (DCM), acetone, Dichloroethane (DCE), and mixtures thereof. More particularly, the solvent is dichloromethane (DCM).

In an embodiment, the present disclosure provides an improved process for the preparation of compound of Formula I as disclosed herein, wherein the substantially pure Nitazoxanide of Formula I in step d) is obtained by precipitation without column chromatography and in step e) after the completion of reaction, the excess of solvent was removed under reduced pressure and the residue was brought down to 0-5° C. and upon isopropanol addition, the substantially pure Nitazoxanide of Formula I is obtained without column chromatography.

The process mentioned above, the steps (a, b and c) to prepare 5-nitrothiazol-2-amine of Formula II are obtained by precipitation without column chromatography and in step (d) the substantially pure Nitazoxanide of Formula I is obtained by precipitation without column chromatography. As disclosed herein, the resultant compound of formula (I) was isolated by eliminating column chromatography. The said isolation may involve purification by washings, filtrations, crystallization, evaporation, etc.

In an embodiment, the present disclosure provides an improved process for the preparation of Nitazoxanide, also known as 2-((5-nitrothiazol-2-yl)carbamoyl)phenyl acetate having the general formula I;

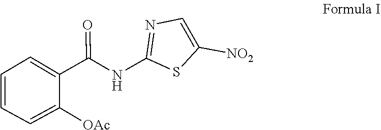

comprising the steps of:
   a) reacting 1,1-dimethoxy-N,N-dimethylethanolamine

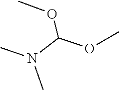

with nitromethane in the presence of an organic solvent selected from selected from the group consisting of tetrahydrofuran (THF), dichloromethane (DCM), ethylene dichloride (DCE), chloroform, benzene, and mixtures thereof, or neat, to obtain N, N-dimethyl-2-nitroethenamine,
b) reacting N, N-dimethyl-2-nitroethenamine

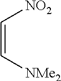

with a brominating reagent selected from the group consisting of bromine or N-bromosuccinimide, in an organic solvent selected from the group consisting of tetrahydrofuran (THF), dichloromethane (DCM), ethylene dichloride (DCE), chloroform, benzene and mixtures thereof to obtain 2-Bromo-N, N-dimethyl-2-nitroethenamine,
c) cyclo condensation of resulting compound 2-Bromo-N, N-dimethyl-2-nitroethenamine

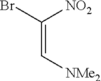

with thiourea in an organic solvent selected from the group consisting of dimethylformamide (DMF), dimethyl sulfoxide (DMSO), dimethylacetamide (DMAc), and mixtures thereof and an acid catalyst selected from the group consisting of acetic acid, sulphuric acid or hydrochloric acid at room temperature to obtain 5-nitrothiazol-2-amine of Formula II,

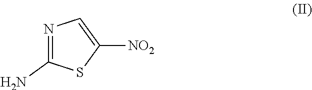

d) condensing the resultant compound of Formula II with 2-acetoxybenzoic acid of Formula III

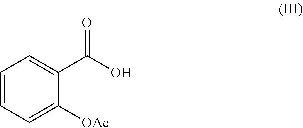 [SMILES: CC(=O)Oc1ccccc1C(=O)O]

in the presence of N—N′-dicyclohexylcarbodiimide (DCC), acetone and triethylamine (Et₃N) as a base to obtain a substantially pure Nitazoxanide of Formula I. and
e) optionally reacting the 2-acetoxybenzoic acid of Formula III,

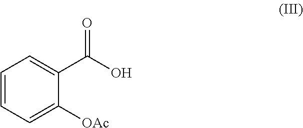 [SMILES: CC(=O)Oc1ccccc1C(=O)O]

with a reagent comprising oxalyl chloride ((COCl)₂) in dimethylformamide (DMF). in an organic solvent selected from the group consisting of tetrahydrofuran (THF), dichloromethane (DCM), acetone, dichloroethane (DCE) and mixtures thereof to obtain 2-(chlorocarbonyl)phenyl acetate of Formula IV,

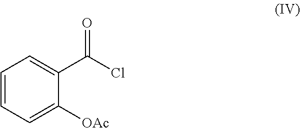 [SMILES: CC(=O)Oc1ccccc1C(=O)Cl]

this upon condensing with 5-nitrothiazol-2-amine of Formula II in the presence of tetrahydrofuran (THF) and triethylamine (Et₃N) as a base to obtain a substantially pure Nitazoxanide of Formula I.

In an embodiment, the present disclosure provides an improved process for the preparation of Nitazoxanide, also known as 2-((5-nitrothiazol-2-yl)carbamoyl)phenyl acetate having the general formula I;

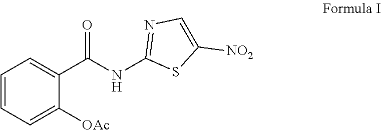

comprising the steps of:
   a) reacting 1,1-dimethoxy-N,N-dimethylethanolamine

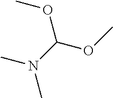

with nitromethane in neat condition, to obtain N, N-dimethyl-2-nitroethenamine,
b) reacting N, N-dimethyl-2-nitroethenamine

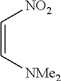

with N-bromosuccinimide as a brominating agent, in an organic solvent selected from the group consisting of chloroform, benzene and mixtures thereof to obtain 2-Bromo-N, N-dimethyl-2-nitroethenamine,
c) cyclo condensation of resulting compound 2-Bromo-N, N-dimethyl-2-nitroethenamine

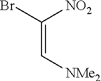

with thiourea in dimethylformamide (DMF), and hydrochloric acid as acid catalyst at room temperature to obtain 5-nitrothiazol-2-amine of Formula II,

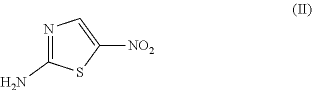

d) condensing the resultant compound of Formula II with 2-acetoxybenzoic acid of Formula III

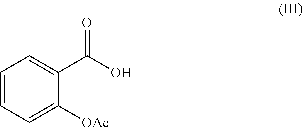 [SMILES: CC(=O)Oc1ccccc1C(=O)O]

in the presence of N—N′-dicyclohexylcarbodiimide (DCC), acetone and triethylamine (Et₃N) as a base to obtain a substantially pure Nitazoxanide of Formula I. and
e) optionally reacting the 2-acetoxybenzoic acid of Formula III,

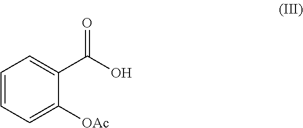 [SMILES: CC(=O)Oc1ccccc1C(=O)O]

with a reagent comprising oxalyl chloride ((COCl)₂) in dimethylformamide (DMF). in dichloromethane (DCM), to obtain 2-(chlorocarbonyl)phenyl acetate of Formula IV,

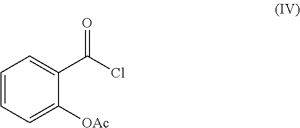 [SMILES: CC(=O)Oc1ccccc1C(=O)Cl]

this upon condensing with 5-nitrothiazol-2-amine of Formula II in the presence of tetrahydrofuran (THF) and triethylamine (Et₃N) as a base to obtain a substantially pure Nitazoxanide of Formula I.

The present invention will now be further explained in the following examples describing in detail the preparation of the said compound of formula (1). However, the present invention should not be construed as limited thereby. One of the ordinary skills in the art will understand how to vary the exemplified preparations to obtain the desired results. The reactions herein disclosed were monitored by TLC (Thin Layer Chromatography) method.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

The following examples, which include preferred embodiments, will serve to illustrate the practice of this invention, it being understood that the particulars shown are by way of example and for purpose of illustrative discussion of preferred embodiments of the invention.

### Example 1

Preparation of Nitazoxanide from Acetylsalicylic Acid

A mixture of 1,1-dimethoxy-N,N-dimethylmethanamine (1.65 g, 14 mmol), and nitromethane (8.66 g, 14 mmol) was heated to 80° C. for 30 minutes by monitoring TLC; after completion of the reaction, the reaction mixture was allowed to cool to room temperature and concentrated directly under reduced pressure. To the resulting N, N-dimethyl-2-nitroethenamine (1.16 g, 10 mmol), the mixture benzene/chloroform (2:4 mL, 1/2, V/V) was added. After dissolving the contents, 1.83 g (10 mmol) of N-bromosuccinimide (NBS) was added under argon atmosphere at 0-5° C. and was stirred for 2 hrs at room temperature. After the completion of the reaction (monitored by TLC), the solvent was removed under reduced pressure and was dried. The obtained residual solid of 2-Bromo-N,N-dimethyl-2-nitroethenamine (1.93 g, 10 mmol) was dissolved in small amounts of DMF, thiourea (1.98 g, 13 mmol) followed by addition of few drops (3-5 drops) of conc. HCl. This solution was stirred at room temperature for 1 hr, the excess of DMF was removed under reduced pressure and was dried to obtain 2-amino-5-nitrothiazole (1.45 g, 13 mmol, Formula II) with a yield of 75%.

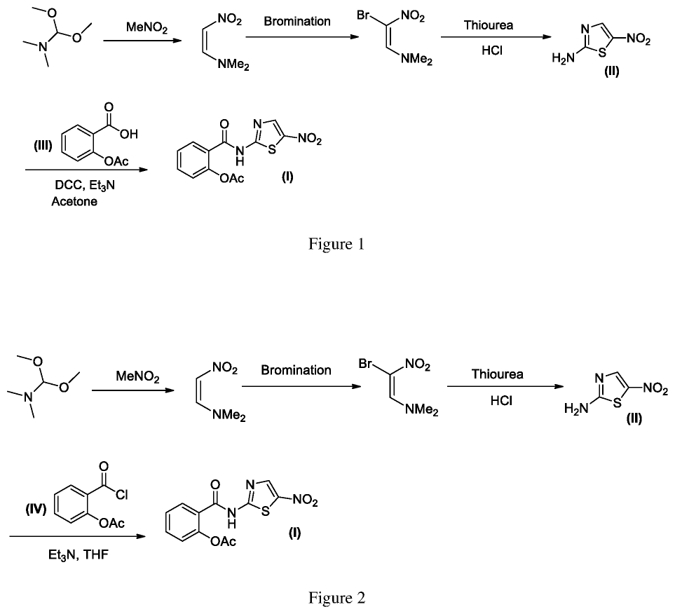

To the same flask containing 2-amino-5-nitrothiazole (1.45 g, 10 mmol), added acetylsalicylic acid (3.62 g, 20 mmol) in dry 10 ml acetone under argon atmosphere, stirred for about 45 minutes at 0-5° C., and after 45 minutes, a solution of N—N′-Dicyclohexylcarbodiimide (6.60 g, 32 mmol) in 20 mL of acetone was added. And also, dry triethylamine (40 mmol) in 10 mL acetone was added drop wise and the reaction mass was stirred for 5 hrs. The reaction mixture concentrated to 5 ml under reduced pressure, was poured into water (40 mL) and was extracted with EtOAc (100 mL×4). The combined organic extract was dried over anhydrous Na₂SO₄, filtered, and washed with EtOAc. Then 1.5 g activated charcoal was added at 50° C. in a hot water bath and colored impurities were removed. The mixture was then filtered while hot and the filtrate was concentrated to 5 mL by removing the solvent under reduced pressure. 20 ml of isopropanol was added to the concentrated solution at 0-5° C. to precipitate Nitazoxanide (Formula I) as a light-yellow solid, which on recrystallization with ethanol yielded 84% of the pure form of Nitazoxanide [FIG. 1].

### Example 2

Preparation of Nitazoxanide from Acetylsalicyloyl Chloride

A mixture of 1,1-dimethoxy-N,N-dimethylmethanamine (1.65 g, 14 mmol) and nitromethane (8.66 g, 14 mmol) was heated to 80° C. for 30 minutes by monitoring TLC; after completion of the reaction, the reaction mixture was allowed cool to room temperature and concentrated directly under reduced pressure. To the resulting N, N-dimethyl-2-nitroethenamine (1.16 g, 10 mmol), a mixture of benzene/chloroform (2:4 mL, 1/2, V/V) was added. After dissolving the contents, 1.83 g (10 mmol) of N-bromosuccinimide (NBS) was added under argon atmosphere at 0-5° C. and was stirred for 2 hrs at room temperature. After the completion of the reaction (monitored by TLC), the solvent was removed under reduced pressure and dried. The obtained residual solid of 2-Bromo-N, N-dimethyl-2-nitroethenamine (1.93 g, 10 mmol) was dissolved in small amounts of DMF, thiourea (1.98 g, 13 mmol) followed by addition of few drops (3-5 drops) of conc. HCl. This solution was stirred at room temperature for 1 hr, the excess of DMF was removed under reduced pressure and was dried to get 2-amino-5-nitrothiazole (1.45 g, 13 mmol, Formula II) with a yield of 75%.

To the same reaction flask containing 2-amino-5-nitrothiazole (1.45 g, 10 mmol) 15 mL of anhydrous tetrahydrofuran (THF) and triethylamine (23 mmol) were slowly added. The reaction mixture was kept under stirring until 2-amino-5 nitrothiazole was completely dissolved. Then under −5° C.˜0° C. temperature, 12.5 mmol of acetylsalicyloyl chloride (2.5 g, Formula IV) in 10 mL of anhydrous THF was slowly added for a time period of 35 min. The reaction mixture was stirred at 45° C. for 5 hours. After completion of the reaction, the reaction mixture was concentrated to 5 mL under reduced pressure. It was poured into water (40 mL), extracted with EtOAc (100 mL×4). The combined organic extract was dried over anhydrous Na₂SO₄, filtered, and washed with EtOAc. Then 1.5 g activated charcoal was added at 50° C. in a hot water bath and the colored impurities were removed. The mixture was then filtered while hot and the filtrate was concentrated to 5 mL by removing the solvent under reduced pressure. 20 ml of isopropanol was added to the concentrated solution at 0-5° C. to precipitate Nitazoxanide compound (Formula I) as a light yellow solid, which on recrystallization with ethanol yielded 78% of the pure form of Nitazoxanide [FIG. 2].

The present invention provides an industrially viable and cost-effective process for the preparation of Nitazoxanide and intermediates thereof. In the prior art methods, synthesis of Nitazoxanide was achieved by utilizing the advanced key starting materials, 2-acetoxybenzoic acid/2-(chlorocarbonyl) phenylacetate and 5-nitrothiazol-2-amine. Whereas, in the present invention, a single vessel process to prepare 5-nitrothiazol-2-amine starting from a very basic starting material 1,1-dimethoxy-N,N-dimethylethanolamine was developed.

The novel single vessel process for the preparation of 5-nitrothiazol-2-amine is the inventive step of the protocol (FIGS. 1 and 2). An improved process for the synthesis of Nitazoxanide from the 5-nitrothiazol-2-amine and 2-acetoxybenzoic acid is another inventive step of the protocol (FIGS. 1 and 2). Overall the process is free from column chromatography, all the intermediates throughout the process and the final product Nitazoxanide purified by precipitation is the one more inventive process step.

### ADVANTAGES OF THE PRESENT INVENTION

The present invention provides an improved process for the preparation of Nitazoxanide by single vessel process, selection of various reaction solvents or precipitation/crystallization techniques, which are useful for the scale up process while retaining the chemical purity of the product.

Present invention relates to a single vessel process by avoiding tedious and time taking multi-step purification process.

Present invention provides an industrially viable and cost effective process for the preparation of pure form of Nitazoxanide.

I/We claim:

1. An improved process for the preparation of Nitazoxanide, also known as 2-((5-nitrothiazol-2-yl)carbamoyl)phenyl acetate having the general formula I; 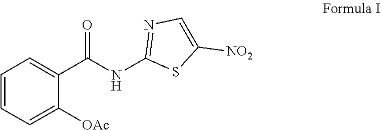
comprising the steps of:
a) reacting 1,1-dimethoxy-N,N-dimethylethanolamine 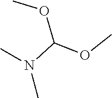
with nitromethane in the presence of an organic solvent or neat to obtain N, N-dimethyl-2-nitroethenamine,
b) reacting N, N-dimethyl-2-nitroethenamine 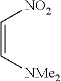
with a brominating reagent in an organic solvent to obtain 2-bromo-N, N-dimethyl-2-nitroethenamine,
c) the cyclo condensation of resulting compound 2-bromo-N, N-dimethyl-2-nitroethenamine 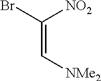
with thiourea in an organic solvent and an acid catalyst to obtain 5-nitrothiazol-2-amine of Formula II, 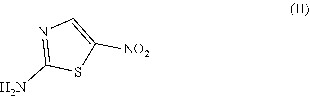
d) condensing the resultant compound of Formula II with 2-acetoxybenzoic acid of Formula III 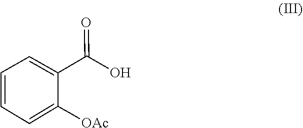 [SMILES: CC(=O)Oc1ccccc1C(=O)O]
in the presence of N—N′-dicyclohexylcarbodiimide (DCC), acetone and triethylamine (Et₃N) as a base to obtain a substantially pure Nitazoxanide of Formula I, and
e) optionally reacting the 2-acetoxybenzoic acid of Formula III, 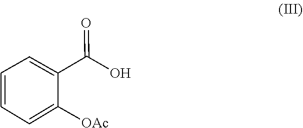 [SMILES: CC(=O)Oc1ccccc1C(=O)O]
with a reagent in an organic solvent to obtain 2-(chlorocarbonyl)phenyl acetate of Formula IV, 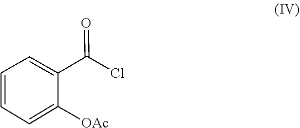 [SMILES: CC(=O)Oc1ccccc1C(=O)Cl]
this upon condensing with 5-nitrothiazol-2-amine of Formula II in the presence of tetrahydrofuran (THF) and triethylamine (Et₃N) as a base to obtain a substantially pure Nitazoxanide of Formula I.

2. The process as claimed in claim 1, wherein in step a) the organic solvent is selected from the group consisting of tetrahydrofuran (THF), dichloromethane (DCM), ethylene dichloride (DCE), chloroform, benzene, and mixtures thereof, or neat.

3. The process as claimed in claim 1, wherein the brominating reagent in step b) is selected from the group consisting of bromine or N-bromosuccinimide.

4. The process as claimed in claim 1, wherein the organic solvent in step b) is selected from the group consisting of tetrahydrofuran (THF), dichloromethane (DCM), ethylene dichloride (DCE), chloroform, benzene and mixtures thereof.

5. The process as claimed in claim 1, wherein the catalyst in step c) is selected from the group consisting of acetic acid, sulphuric acid or hydrochloric acid; and the solvent in step c) is selected from the group consisting of dimethylformamide (DMF), dimethyl sulfoxide (DMSO), dimethylacetamide (DMAc), and mixtures thereof.

6. The process as claimed in claim 1, wherein the step c) to convert 2-bromo-N, N-dimethyl-2-nitroethenamine into 5-nitrothiazol-2-amine of Formula II is carried out at room temperature.

7. The process as claimed in claim 1, wherein the reagent in step e) for conversion of compound of Formula III to compound of Formula IV is oxalyl chloride ((COCl)₂) in dimethylformamide (DMF).

8. The process as claimed in claim 1, wherein the solvent in step e) for conversion of compound of Formula III to Formula IV, is selected from the group consisting of tetrahydrofuran (THF), dichloromethane (DCM), acetone, dichloroethane (DCE) and mixtures thereof.

9. The process as claimed in claim 1, wherein all the steps (a, b c) to prepare 5-nitrothiazol-2-amine of Formula II is obtained by precipitation without column chromatography.

10. The process of as claimed in claim 1, wherein the substantially pure Nitazoxanide of Formula I in step d) is obtained by precipitation without column chromatography and in step e) after the completion of reaction, the excess of solvent was removed under reduced pressure and the residue was brought down to 0-5° C. and upon isopropanol addition, the substantially pure Nitazoxanide of Formula I is obtained without column chromatography.
